(19) United States
(12) Patent Application Publication
(10) Pub. No.: US 2025/0302492 A1
(43) Pub. Date: Oct. 2, 2025

(54) METHOD AND SYSTEM FOR SHORTENING A BONE

(71) Applicant: TriMed, Inc., Santa Clarita, CA (US)

(72) Inventor: Robert J. Medoff, Kailua, HI (US)

(21) Appl. No.: 19/048,522

(22) Filed: Feb. 7, 2025

Related U.S. Application Data
(60) Provisional application No. 63/570,026, filed on Mar. 26, 2024.

(51) Int. Cl. A61B 17/17

(52) U.S. Cl. CPC A61B 17/1775; A61B 17/171

(57) ABSTRACT

A method and a guide system for performing a bone osteotomy. The guide system has a block defining first and second edges each for guiding a cutting component. The block and a cooperating base each is configured to be releasably fixed in an operative position relative to a bone. The block has a connector that cooperates with a connector on the base to guide relative movement of the base and block substantially along a first line. Separation of a bone fragment formed using the guided cutting component produces a gap of a first width between facing surfaces on relatively movable bone parts, with the base and block fixed one each thereto. The block and base are configured so that with the bone fragment separated, the block and base can be guidingly relatively moved to thereby: a) reduce the width of the gap from the first width; b) move the facing surfaces towards each other; and c) place the bone parts in a desired relationship. The bone parts can be fixed to each other in different manners to maintain the desired relationship.

### CROSS-REFERENCE TO RELATED APPLICATIONS

This is a non-provisional application claiming priority to U.S. Provisional Application No. 63/570,026, filed Mar. 26, 2024, which is incorporated herein by reference.

### BACKGROUND OF THE INVENTION

### Field of the Invention

This invention relates to a method of shortening, and system usable to shorten, a bone to address any of a number of structural issues associated therewith.

### Background Art

Bone shortening osteotomies are performed to address a number of structural issues. Without limitation, a metatarsal shortening osteotomy is commonly performed with at least one of the objectives of decreasing pain at the base of a second toe and reducing excessive load on a long second toe. Shortening a distal ulna is another common procedure with an objective of decreasing pain related to excessive bone length.

Metatarsal shortening osteotomies, especially when performed at the distal end of the bone, are inherently challenging to perform for a number of reasons. First, the second metatarsal is a relatively small bone which limits how it can be controlled during a procedure and what types of procedures may be performed. Additionally, the second metatarsal is supported by soft tissue which provides little stabilization during a shortening osteotomy. Additionally, it is difficult to: a) precisely determine the amount of shortening; b) visualize the osteotomy; and c) position and hold the unstable fragment/part in accurate apposition during fixation due to adjacent metatarsal bones that limit access.

The ability to precisely separate a metatarsal bone fragment, control relatively movable bone parts that are produced by the removed bone fragment, and strategically place one or more fixation screws remains a challenge to surgical equipment designers and surgeons to this day.

One known procedure is identified as a “Weil osteotomy.” With this technique, a surgeon makes a substantially horizontal cut, which is slightly oblique, and thereafter slides the separated distal part over a 2-3 mm distance against the proximal part, after which a fastening screw is inserted.

Because the cut is slightly oblique, as the distal part is shifted it also displaces plantarly, which results in tension being applied on the toe extensors and the toe flexors becoming relaxed. This can cause the toe to cock up, which is an undesirable result. Instability of the distal fragment/bone part and inability to directly observe the reduction of bone fragments can often result in misplacement of fixation or unfavorable apposition, resulting in poor reduction and/or fixation. Complications from the procedure are common.

Surgeons have generally contended with the above challenges due to the absence of practical alternatives. That is, surgeons have struggled to establish and maintain alignment of relatively movable bone parts resulting from the bone cutting—be it to remove a fragment or create a horizontal separation line. Slight misalignment may compromise the efficacy of the procedure. Further, direction of a fastening screw only a couple millimeters off of an optimal path may result in a fastener screw cracking through the edge of one or more of the bone parts.

Existing systems used to assist the carrying out of procedures on larger bones are generally not adaptable to be used with bones as small as second metatarsal bones. Thus, the various steps may be performed in “freehand” fashion, without the assistance of precision guidance and stabilization of relatively movable bone parts as they are moved into desired alignment preparatory to inserting fastener screws that maintain the desired final relationship of the bone parts.

### SUMMARY OF THE INVENTION

In one form, the invention is directed to a method of shortening a bone. The method includes the steps of: obtaining a guide system having a block and a base; releasably fixing the block and base in a first operative relationship with each other and the bone; with the block and the base in the first operative relationship with each other and the bone, using the block to guide a cutting component relative to the bone so as to cut the bone to thereby form a separable bone fragment between first and second relatively movable bone parts; separating the bone fragment from the bone so that a gap is defined between facing surfaces on the first and second relatively movable bone parts; after separating the bone fragment, relatively moving the base and block to thereby cause the first and second bone parts to relatively move so that the facing surfaces on the first and second bone parts are moved closer to each other to thereby place the first and second bone parts in a desired relationship; fixing the first and second bone parts in the desired relationship; and separating the block and base from the bone. With the block and base fixed in the first operative relationship with each other and the base: a) the block is fixed to a part of the bone, that becomes the first bone part, through at least a first fixation component; and b) the base is fixed to a part of the bone, that becomes the second bone part, through at least a second fixation component.

In one form, the step of fixing the first and second bone parts involves fixing a component to each of the first and second bone parts.

In one form, the step of fixing the first and second bone parts involves directing at least one fastener through the component and into each of the first and second bone parts.

In one form, the step of fixing the first and second bone parts involves engaging a part of a component with the first bone part and directing at least one fastener through the component and into the second bone part.

In one form, the step of fixing the first and second bone parts involves directing a fixation element into the first and second bone parts.

The bone has a length. In one form, the step of directing a fixation element involves directing a first fixation element lengthwise of the bone into each of the first and second bone parts.

In one form, the step of directing a first fixation element lengthwise of the bone involves directing the fixation element through an intramedullary space on at least one of the first and second bone parts.

In one form, the step of directing a fixation element into the first and second bone parts involves directing at least a first fixation element through the facing bone surfaces.

In one form, the bone is one of an ulna bone and a second metatarsal bone.

In one form, the guide system further includes a first guide block that defines at least a first guide passage. The method further includes the step of directing a drilling component through the first guide passage to define an advancement line for the first fixation element.

In one form, the method further includes the step of operatively engaging the first guide block with at least one of the block and base.

In one form, the guide system further includes first and second different guide blocks each defining a guide passage. The method further includes the steps of operatively engaging a selected one of the first and second different guide blocks with at least one of the block and base and thereafter directing a drilling component through the guide passage on the selected one of the first and second different guide blocks to define an advancement line for the first fixation element. The first and second different guide blocks are interchangeably usable, one in place of the other.

In one form, the guide system further includes a first guide block that defines first and second guide passages. The method further includes the step of directing a first drilling component through one of the first and second guide passages to define an advancement line for the first fixation element.

In one form, the block has first and second spaced guiding edges which guide at least one of: a) a part of a cutting tool having the cutting component; and b) the cutting component.

In one form, there is a single piece that defines the first and second guiding edges and a guide passage that defines an advancement line for the first fixation element.

In one form, the block has a connector that cooperates with a connector on the base. The step of relatively moving the base and block involves guidingly moving the base and block through the cooperating connectors on the base and block.

In one form, the connector on one of the block and base is in the form of a rail that moves guidingly within a slot making up the connector on the other of the block and base such that the block and base are relatively movable along a substantially straight line and are keyed against turning angularly relative to each other around the substantially straight line.

In one form, the method further includes the step of fixing the first operative relationship between the block and base using a fastener cooperating between the block and base.

In one form, the method further includes the step of fixing a second relationship between the block and base through a fastener cooperating between the block and base with the first and second bone parts in the desired relationship.

In one form, the fastener that cooperates between the block and base with the block and base in the first operative relationship is the same fastener that cooperates between the block and base with the block and base in the second relationship, and is a threaded fastener that is directed into: a) a first receptacle on the block with the block and base in the first operative relationship with each other and the bone; and b) a second receptacle on the block with the block and base in the second relationship.

In one form, the fastener that cooperates between the block and base with the block and base in the first relationship is a same fastener that cooperates between the block and base with the block and base in the second relationship and is a threaded fastener that is directed into a first receptacle on the block with: a) the block and base in the first operative relationship; and b) with the block and base in the second relationship.

In one form, the threaded fastener extends through: a) a first receptacle on the block with the block and base in the first operative relationship; and b) a second receptacle on the block with the block and base in the second relationship.

In one form, the step of releasably fixing the block and base in the first operative relationship involves tightening the fastener. The method further includes the step of loosening the tightened fastener with the block and base in the first operative relationship and without separating the fastener from the base, after loosening the tightened fastener, relatively moving the block and base from the first operative relationship into the second relationship, and re-tightening the fastener to fix the second relationship between the block and base.

In one form, the block has another connector that cooperates with another connector on the base and each of the: a) cooperating connectors; and b) cooperating another connectors is in the form of a rail that moves guidingly within a slot.

In one form, the bone has a length. The cooperating connectors are spaced from the cooperating another connectors along the length of the bone.

In one form, the step of relatively moving the base and block involves using a clamping device to facilitate relative movement of the first and second bone parts so as to thereby urge the facing bone surfaces towards each other.

In one form, the step of releasably fixing the block and base in the first operative relationship involves directing first and second drilling components through the block into the bone.

In one form, the step of releasably fixing the block and base involves directing first and second drilling components through the base into the bone.

In one form, the bone has a length. The step of relatively moving the base and block involves causing the first and second bones to relatively move along a path substantially parallel to the length of the bone.

In one form, the fastener guides movement between the block and base as the block and base are changed between the first operative relationship and the second relationship.

In one form, the bone has a surface against which the block is placed with the block and base in the first operative relationship with each other and the bone. The first and second guiding edges are substantially parallel and make an angle of 25°-45° with respect to a length of the bone.

In one form, the bone fragment has a substantially uniform thickness.

In one form, with the block and base in the first operative relationship with each other and the bone, the block bears directly against the first bone part and the base bears directly against the second bone part.

In one form, the base has first and second spaced, elongate, depending portions that each bears against the second bone part with the block and base in the first operative relationship with each other and the bone.

In one form, the block has first and second spaced guiding edges which guide at least one of: a) a part of a cutting tool having the cutting component; and b) the cutting component, the block has spaced ends, and each of: i) the first and second spaced guiding edges on the block; and ii) the first receptacle on the block are closer to a same one of the spaced ends on the block.

In one form, the invention is directed to a guide system for performing an osteotomy on a bone. The guide system includes a block and a base. The block has first and second edges each for guiding a cutting component in a controlled path. The block is configured to be placed in an operative position against the surface of the bone and to be releasably fixed in the operative position. The base is configured to be placed in an operative position against the surface of the bone and to be releasably fixed in its operative position. The block has a connector that cooperates with a connector on the base to guide relative movement between the base and block substantially along a first line. The block and base are configured so that in respective operative positions a cutting component can be guided through the first and second edges to produce a separable bone fragment. Separation of the bone fragment produces a gap of a first width between facing surfaces on relatively movable bone parts. The block and base are configured so that with the bone fragment separated, the block and base can be guidingly relatively moved to reduce the width of the gap from the first width, move the facing surfaces towards each other, and place the relatively movable bone parts in a desired relationship.

In one form, the guide system is provided in combination with at least one drilling component that can be directed through the block and into the bone to maintain the block in its operative position.

In one form, the guide system is provided in combination with at least one drilling component that can be directed through the base and into the bone to maintain the base in its operative position.

In one form, the guide system further includes a fastener that cooperates between the base and block to selectively releasably maintain the base and block in first and second different relative positions.

In one form, the fastener is a threaded fastener that can be directed into at least one receptacle on the block.

In one form, the block has a single receptacle for the threaded fastener. The base has first and second receptacles through which the threaded fastener is selectively directed and into the single receptacle on the block. The guide system is configured so that the threaded fastener can be: a) loosened without being separated from the base to allow the block and base to be relatively moved between the first and second different relative positions wherein the threaded fastener is respectively directed through the first and second receptacles on the base; and b) re-tightened to releasably maintain the block and base in the first and second different relative positions.

In one form, the cooperating connectors are on the block and base and make up a rail and slot connection whereby the block and base are guided relative to each other.

In one form, the block has another connector that cooperates with another connector on the base and each of the: a) cooperating connectors; and b) cooperating another connectors is in the form of a rail that moves guiding within a slot.

In one form, the bone has a length. The cooperating connectors are spaced from the cooperating another connectors along the length of the bone.

In one form, there is a guide passage on at least one of the block and base that defines an advancement line for a fixation element that can be directed through the bone parts and the facing bone surfaces to maintain the bone parts in the desired relationship.

In one form, the block has a body that defines the first and second edges and the guide passage.

In one form, the guide passage is defined on a first guide block that is configured to be releasably engaged with at least one of the block and base.

In one form, the guide system is provided in combination with a second guide block with a guide passage that is different than the first guide block and that is configured to be releasably engaged with at least one of the block and base. The first and second guide blocks are interchangeably usable, one in place of the other.

In one form, with the first and second guide blocks engaged with the at least one of the block and the base, the guide passages on the first and second guide blocks are different by defining advancement lines having at least one of: a) different orientations; and b) different entry locations on the bone.

In one form, the guide system is provided in combination with a component usable to maintain the bone parts in the desired relationship.

In one form, the component is configured to be directed into an intramedullary space of one of the first and second bone parts.

In one form, the fastener is configured to guide relative movement of the base and block between the first and second different relative positions.

In one form, the guide system is provided in combination with at least one drilling component configured to be directed through the base and into a bone to releasably fix the base in its operative position.

In one form, the guide system is provided in combination with at least one drilling component configured to be directed through the block and into a bone to releasably fix the block in its operative position.

### BRIEF DESCRIPTION OF THE DRAWINGS

FIG. 1 is a schematic representation of a guide system for performing a shortening osteotomy on a bone, according to the present invention;

FIG. 2 is a schematic depiction of a cutting tool used to cut bone to produce a separable bone fragment;

FIG. 3 is a schematic representation of bones with which the inventive guide system can be utilized;

FIG. 4 is a schematic depiction of a drilling component utilized in conjunction with the guide system in FIG. 1;

FIG. 5 is a schematic representation of a method of shortening a bone, according to the present invention;

FIG. 6 is a plan view of one form of the block, as identified schematically on the system in FIG. 1;

FIG. 7 is a cross-sectional view of the block taken along line 7-7 of FIG. 6;

FIG. 8 is an end elevation view of the block in FIGS. 6 and 7;

FIG. 9 is an elevation view of the block in FIGS. 6-8 from the end opposite that in FIG. 8;

FIG. 10 is a plan view of one form of base, as shown schematically in FIG. 1;

FIG. 11 is a side elevation view of the base in FIG. 10;

FIG. 12 is an end elevation view of the base in FIGS. 10 and 11;

FIG. 13 is a top view of a fastener used to maintain one or more relationships between the block in FIGS. 6-9 and the base in FIGS. 10-12;

FIG. 14 is an elevation view of the fastener in FIG. 13;

FIG. 15 is a plan view of one exemplary form of guide block as shown schematically in FIG. 1 and engageable with the block, also shown in FIG. 1;

FIG. 16 is a side elevation view of the guide block in FIG. 15;

FIG. 17 is a view as in FIG. 7 with the fastener in FIGS. 13 and 14 directed into one receptacle on the block;

FIG. 18 is a view as in FIG. 17 wherein the base in FIGS. 10-12 is also shown in cross-section and connected to the block;

FIG. 19 is a fragmentary, side elevation view of a bone with the base and block as shown in FIG. 18 releasably fixed in a first operative relationship with each other and the bone;

FIG. 20 is a view as in FIG. 19 wherein a cutting component is shown being guided to cut the bone to produce a separable bone fragment and form relatively movable first and second bone parts;

FIG. 21 is a view as in FIG. 20 wherein the bone fragment is being removed;

FIG. 22 is a view as in FIG. 21 wherein the fastener in FIGS. 13 and 14 is separated to allow the relationship between the block and base to be changed;

FIG. 23 is a view as in FIG. 22 wherein the block and base, not shown in cross-section, have been relatively moved through a clamping tool to close a gap created by the separated bone fragment and with the fastener of FIGS. 13 and 14 in place to maintain another relationship of the block and base and in turn the desired relationship of the relatively movable first and second bone parts resulting from the removal of the bone fragment;

FIG. 24 is a view as in FIG. 23 wherein one form of guide block, as shown schematically in FIG. 1, is guided by the block into an operative position;

FIG. 25 is a view as in FIG. 24 wherein drilling components are directed through guide passages on the guide block which establish advancement lines through the bone parts;

FIG. 26 is a view as in FIG. 25, with the block sectioned as in FIG. 7, and wherein the guide block has been removed and a separate drilling component is guided with respect to the drilling components in FIG. 25 to produce bores for fasteners;

FIG. 27 is a view of the bone as in FIG. 26 wherein the block, base, and drilling components have been separated and fasteners have been directed into the bores along the established advancement lines to fix a desired relationship between the first and second bone parts;

FIG. 28 is an enlarged, perspective view of an exemplary fastener as used in FIG. 27;

FIG. 29 is a view similar to that in FIG. 27 from an opposite side of the bone and with a washer interposed between heads of the fasteners and the bone;

FIG. 30 is a schematic representation of an alternative form of guide block that can be used interchangeably with the guide block as shown in FIGS. 15, 16, 24, and 25;

FIG. 31 is a schematic representation of a component that may be used to fix a desired relationship between the first and second bone parts;

FIG. 32 is a schematic representation of one exemplary form of component as shown in FIG. 31;

FIG. 33 is a schematic representation of another form of component as shown in FIG. 31;

FIG. 34 is a side view of first and second bone parts with an exemplary form of a component as shown in FIG. 33 for fixing a desired relationship between the first and second bone parts;

FIG. 35 is a view as in FIG. 34 and showing in schematic form another form of the component in FIG. 33;

FIG. 36 is a perspective view of another form of guide system, according to the invention and as shown schematically in FIG. 1;

FIG. 37 is an exploded view of the guide system in FIG. 36;

FIG. 38 is an exploded view as in FIG. 37 and from a different perspective;

FIG. 39 is an exploded view as in FIGS. 37 and 38 and from a different perspective;

FIG. 40 is an exploded view as in FIGS. 37-39 and from a still further different perspective;

FIG. 41 is an enlarged, perspective view of a guide block on the guide system in FIGS. 36-40;

FIG. 42 is a perspective view of a guide system, as in FIGS. 36-40, with a modified form of guide block;

FIG. 43 is a perspective view of another form of guide system, according to the invention and as shown schematically in FIG. 1;

FIG. 44 is a perspective view of the guide system in FIG. 43 and from a different perspective;

FIG. 45 is an end elevation view of the guide system in FIGS. 43 and 44;

FIG. 46 is an exploded perspective view of the guide system in FIGS. 43-45;

FIG. 47 is a perspective view of the guide system in FIGS. 43-46 and with a guide block, shown in FIGS. 43-46, removed;

FIG. 48 is an end elevation view of the components in FIG. 47;

FIG. 49 is a perspective view of a block on the guide system in FIGS. 43-48;

FIG. 50 is a plan view of the block in FIG. 49;

FIG. 51 is a bottom view of the block in FIGS. 49 and 50; and

FIG. 52 is a perspective view of a base that cooperates with the block in FIGS. 49-51, as shown in FIGS. 43-48.

### DETAILED DESCRIPTION OF THE PREFERRED EMBODIMENT

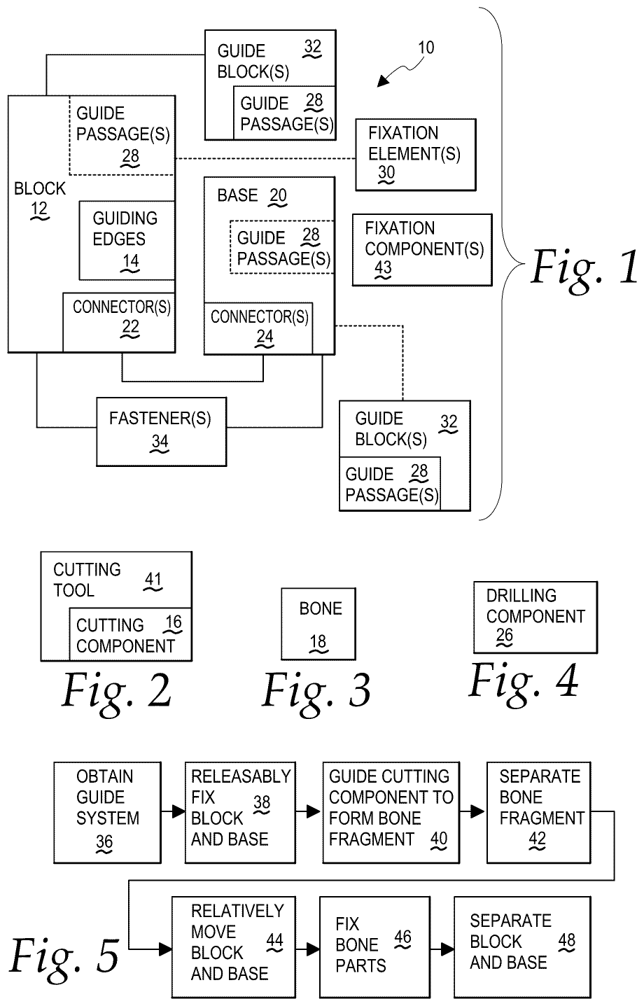

FIG. 1 is a schematic depiction of a guide system 10, according to the present invention, for performing a shortening osteotomy on a bone. The guide system 10 includes a block 12 defining multiple edges 14 for guiding a cutting component 16, as shown schematically in FIG. 2, in separate controlled paths.

The block 12 is configured to be placed in an operative position against the surface of a bone, as shown schematically at 18 in FIG. 3. The bone 18 may be any bone typically shortened by cutting the bone so as to produce a separable fragment after which the parts of the bone 18, that become relatively movable by reason of the fragment separation, are moved into and maintained in, a desired end relationship which effectively shortens the overall length of the bone 18. In one form, the bone 18 may be an ulna bone. For purposes of example only, and without limitation, the focus herein will be upon the second metatarsal bone. The inventive concepts, when practiced with the second metatarsal bone, may overcome those problems identified in the Background Art section, above.

The block 12 is configured to be releasably fixed in the aforementioned operative position.

A base 20 is configured to be placed in an operative position against the surface of the bone and can be releasably fixed in its operative position.

The block 12 has at least one connector 22 that cooperates with at least one connector 24 on the base 20 so as to guide relative movement between the block 12 and base 20 in a controlled path, and as described herein substantially along a first line.

The block 12 and base 20 are configured so that when in respective operative positions, the cutting component 16 can be guided through first and second of the edges 14 to produce a separable fragment from the bone 18.

Regardless of the precise configuration of the bone fragment that is formed, in terms of shape and thickness, upon separation of the bone fragment, a gap with a first width is defined between facing surfaces on the relatively movable bone parts, with the block 12 remaining fixed to one of the relatively movable bone parts and the base 20 remaining fixed to the other of the relatively movable bone parts.

With the bone fragment separated, the block 12 and base 20 are configured to be guidingly relatively moved to reduce the aforementioned gap width by strategically moving the facing bone surfaces towards each other to thereby cause the relatively movable bone parts to be placed in a desired relationship.

At least one drilling component 26, as shown schematically in FIG. 4, may be used to releasably fix/maintain each of the block 12 and base 20 in its respective operative position.

As used herein, a “drilling component” 26 is intended to encompass virtually any type of component that can be directed into bone as to be releasably, or fixedly, held therein. For example, the drilling component 26 may be a conventional K-wire, a component that is turned to form a bore, a threaded component such as a threaded fastener, etc.

In one exemplary form, with one or more drilling components 26 strategically directed through each of the block 12 and base 20 and into the bone 18, the block 12 and base 20 can thereby be releasably maintained in their respective operative positions.

At least one guide passage 28 is provided on the block 12. Each guide passage 28 has an axis along which a fixation element 30 is ultimately aligned. The axis defines an advancement line for each drilling component including, in one form, each fixation element 30 extending through the aforementioned relatively movable bone parts and the facing bone surfaces to maintain the bone parts in the desired end relationship.

The guide passage(s) 28 may be an integral part of the block 12, as shown in dotted lines, and/or may be formed in one or more joinable guide blocks 32 that can be engaged with the block 12.

For example, the block 12 may have a body that defines the guiding edges 14 as well as one or more guide passages 28. In that case, the guide block may be considered to be an integral part of the block 12.

Alternatively, or additionally, as shown in dotted lines in FIG. 1, one or more guide passages 28 may be formed on the base 20. The guide passage(s) 28 may be integrally formed on the base 20 and/or may be formed in one or more guide blocks 32 that can be engaged with/attached to the base 20.

With the separate guide block(s) 32, each guide block 32 may be releasably engaged with the block 12 and/or base 20, or joined thereto and fixedly held. In the former case, guide blocks 32 with different configurations may be utilized interchangeably, as described in greater detail hereinbelow.

At least one fastener 34 cooperates between the block 12 and base 20 to selectively and releasably fix/maintain the base 20 and block 12 in different relative positions.

The components making up the guide system 10 in FIG. 1, as well as the components in FIGS. 2-4 that interact therewith, are shown in schematic form to encompass virtually an unlimited number of different forms of each of the components and their interactions. The invention is not limited to the specific exemplary forms described hereinbelow.

The invention is also directed to a method of shortening a bone as shown schematically in FIG. 5.

As shown at block 36, a guide system, such as that depicted in FIG. 1, is obtained, including a block and base.

As shown at block 38, the block and base are releasably fixed in a first operative relationship with each other and a bone.

As shown at block 40, with the block and base in the first operative relationship with each other and the bone, the block is used to guide a cutting component relative to the base so as to form a separable bone fragment. The cutting component 16 in FIG. 2 may be directly engaged with the edges 14 or a part of a cutting tool 41 that operates the cutting component 16 may be guided directly against the edges 14.

As shown at block 42, the bone fragment is separated from the bone so that a gap of a first width is defined between facing surfaces on first and second relatively movable bone parts.

In one form of carrying out the fixation step shown at block 38, at least a first fixation component, as shown schematically at 43 in FIG. 1, and which may be in the form of the aforementioned drilling component, is extended into the first bone part and engages the block 12, and at least a second of the fixation components 43 is extended into the second bone part and engages the base 20. The fixation components, as schematically depicted, encompass other structures that fix the block 12 and base 10 to respective bone parts, potentially without extending through the block 12 and/or base 20 and/or into one or both of the bone parts.

As shown at block 44, after separating the bone fragment, the base 20 and block 12 are relatively moved to thereby cause the separate first and second bone parts, fixed one each thereto, to also relatively move so that the facing surfaces on the first and second relatively movable bone parts are moved closer to each other to thereby place the first and second bone parts in a desired end relationship. The aforementioned gap is thereby reduced in width from the first width.

As shown at block 46, the first and second bone parts are fixed to maintain the first and second bone parts in the desired end relationship. This fixation may be effected in many different manners, using different structures/components, with exemplary forms described hereinbelow.

As shown at block 48, the block 12 and base 20 are subsequently separated from the bone 18.

Again, the schematic depiction of the method steps is intended to encompass those specifically described herein as well as virtually an unlimited number of variations thereof that may be performed using different structure and/or techniques. The methods described below are exemplary in nature only and should not be viewed as limiting.

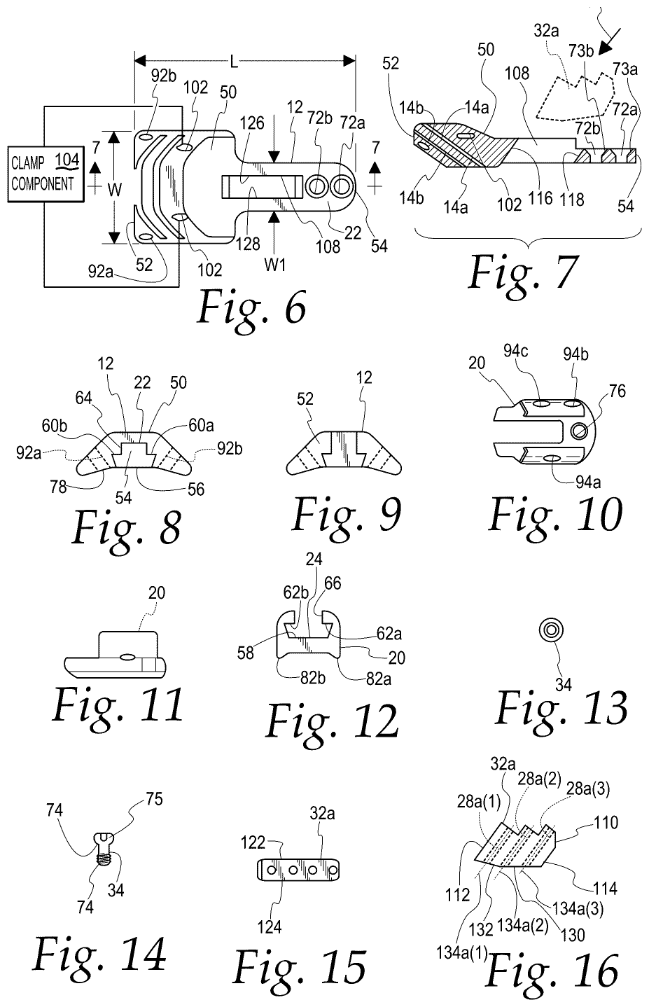

Referring now to FIGS. 6-30, one exemplary form of the guide system 10 and an exemplary form of the inventive method, each at least generally within the schematic depictions in FIGS. 1-5, will be described.

The block 12 has a body 50 with a length L between spaced ends 52, 54. From a plan perspective, the body 50 has a generally squared “T” shape with a width W at the end 52 that narrows mid-length to a width W1 that continues to the end 54.

The portion of the body 50 with the narrower width W1 defines the aforementioned connector 22 on the block 12 that cooperates with the connector 24 on the base 20. The cooperating cross-sectional configurations of the connectors 22, 24 can be seen most clearly in FIGS. 8 and 12, understanding that the connector forms can be reversed on the block 12 and base 20.

In this embodiment, the connector 22 is in the form of a rail, with the connector 24 having a complementary slot configuration. The connector arrangement produces a captive rail and slot configuration. The connector 22 has an inverted “T” shape, with the top surface 56 of the “T” confronting an upwardly facing surface 58 on the base 20. The crossbar of the “T” shape on the connector 22 has spaced surfaces 60a, 60b which respectively confront surfaces 62a, 62b on the base 20. The stem 64 of the “T” moves within a complementary opening 66 on the base 20.

With this arrangement, the base 20, starting from a fully spaced position as shown in dotted lines in FIG. 18, can be situated to align the rail-and-slot configurations on the connectors 22, 24, whereupon the base 20 can be advanced in the direction of the arrow 68 in FIG. 18 to cause the rail connector 22 to move into the slot connector 24. The base 20 and block 12 are guidingly movable, one against the other, in a translational/substantially straight line path as indicated by the double-headed arrow 70 in FIG. 18. This slot-and-rail arrangement keys the base 20 and block 12 against turning angularly relative to each other around the straight line path along which they are relatively movable. That is, the only substantial relative movement allowed between the block 12 and base 20 is translational.

Different relationships between the base 20 and block 12 can be fixed using the aforementioned fastener 34, shown clearly in FIGS. 13 and 14 as a threaded coupling screw 34.

To maintain two predetermined relationships between the block 12 and base 20, with it understood that any number of relationships might be maintained, first and second receptacles 72a, 72b are formed in the block body 50. The receptacles 72 are shown as through openings with enlarged, beveled portions 73a, 73b to seat a complementarily-shaped surface 74 on the head 75 of the fastener 34.

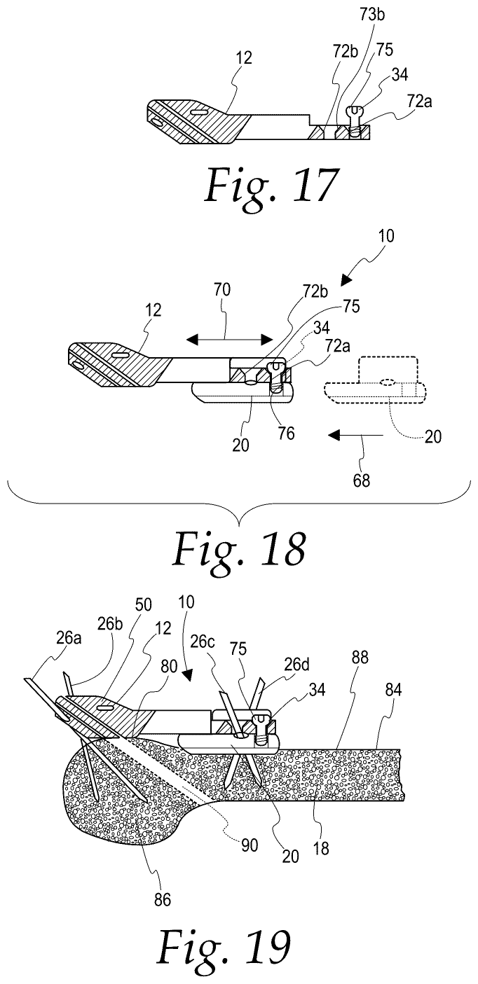

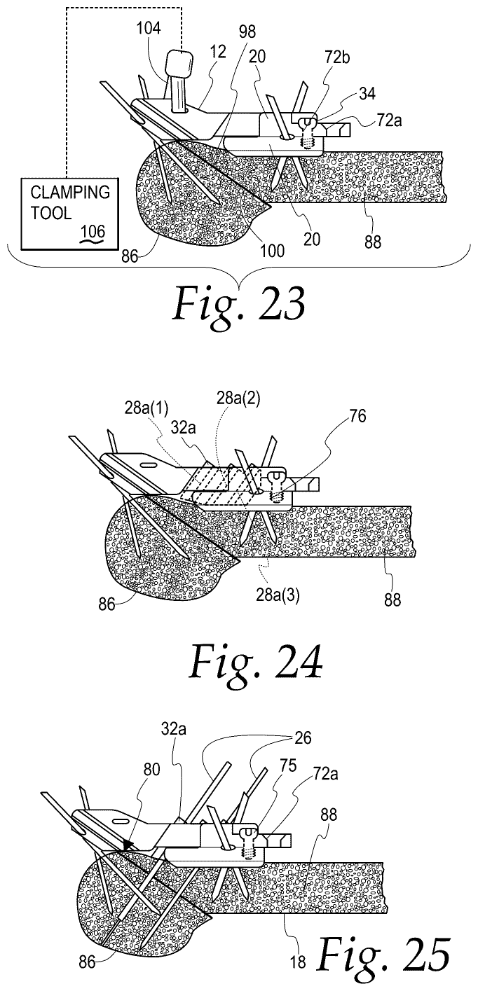

A threaded bore 76 on the base 20 is selectively registrable with the receptacles 72a, 72b. As shown in FIGS. 17 and 18, with the base 20 and block 12 in a first relationship, the fastener 34 can be passed through the receptacle 72a and threaded into the bore 76. With the base 20 shifted relative to the block 12 in a leftwise direction as shown in FIGS. 23-25, the receptacle 72b is aligned with the bore 76. This second relationship can be maintained by directing the fastener 34 through the receptacle 72b and threadably engaging the fastener 34 in the bore 76.

As seen clearly in FIG. 7, the body 50 is thicker adjacent to the end 52 at which the edges 14 are defined. As depicted, the cutting edges 14 are arranged to allow guiding of the cutting component 16 to produce parallel cut lines. The precise arrangement—spacing and orientation—of the edges 14 is not critical. As depicted, the cutting component 16 will be guided between facing edge pairs 14a, 14a and 14b, 14b.

The underside of the block body 50 has a concave surface portion 78 to nominally match and seat against the enlarged distal region 80 of the exemplary metatarsal bone 18, as seen in FIG. 19.

The base 20 has spaced, elongate guide feet 82a, 82b which, with the block 12 and base 20 connected, project to below the block 12, as seen in FIGS. 18 and 19, with this shape accommodating the smaller diameter of the bone 18 directly engaged by the base 20. The spaced foot arrangement produces a straddling engagement between the base 20 and bone with spaced contact regions and thereby stably supports the base 20 against the exposed bone surface 84, as seen in FIGS. 17-26, while at the same time producing a gripping action at the bone surface 84 that resists shifting of the base 20 along the length of the bone 18 and around the length of the bone 18.

To initiate a procedure, the base 20 and block 12 are releasably fixed in a first operative relationship with each other and the bone 18. This first operative relationship is shown in FIG. 19, with the underside of the block 12 and the base feet 82 bearing against the exposed bone surface 84. The fastener 34 is directed through the receptacle 72a on the body 50 and into the bore 76 on the base to maintain the relationship between the base 20 and block 12. This first operative relationship is further maintained by directing, in this case, two drilling components 26a, 26b guidingly through the block 12 and into the bone 18 and at least two additional drilling components 26c, 26d guidingly through the base 20 and into the bone 18. As depicted, and without limitation, the exemplary drilling components 26 are K-wires.

As seen in FIG. 19, the first operative relationship between the block 12, base 20, and bone 18 is selected so that the drilling components 26a, 26b will be directed into a part of the bone 18 that will become the aforementioned first bone part 86, with the drilling components 26c, 26d each entering a part of the bone 18 that will become the aforementioned second bone part 88, such that the bone parts 86, 88 become relatively movable, respectively together with the block 12 and base 20, upon removing the bone fragment 90, identified with dotted lines in FIG. 19.

Further, the drilling components 26a, 26b are strategically directed at angles to positively hold the block 12 against shifting relative to the surface 84 on the bone part 86 that it abuts. Likewise, the drilling components 26c, 26d are strategically angled so that they, together with the guide feet 82, resist shifting of the base 20 relative to the bone 18.

For strategic placement, alignment and controlled guided movement of the drilling components 26, through bores 92a, 92b may be provided on the body 50 with axes aligned to produce the desired bone entry location and angle of the drilling components 26a, 26b, respectively.

Likewise, strategically located and aligned through bores 94a, 94b accommodate the drilling components 26c, 26d, respectively. An additional through bore 94c is provided on the base 20.

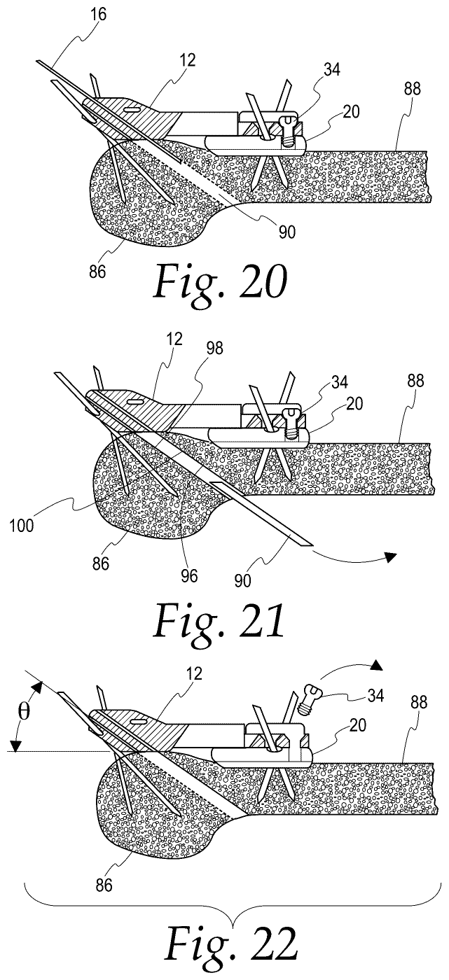

Once the block 12 and base 20 are releasably fixed in the first operative relationship with each other and the bone 18, as shown in FIG. 19, the schematically depicted cutting component 16 may be guided separately along the guiding edges 14 to form the separable bone fragment 90, as shown in FIG. 20. The edges 14 are shown to be substantially parallel to produce a bone fragment of substantially uniform thickness. However, this parallel relationship is not required.

As shown in FIG. 21, the bone fragment 90 is thereafter separated, which results in the formation of a gap 96 of a first width between facing surfaces 98, 100, respectively on the first and second bone parts 86, 88. At this stage, the block 12 remains firmly fixed to the first bone part 86, with the base 20 firmly fixed to the second bone part 88 and also fixed in relationship to the block 12 through the fastener 34.

As depicted in FIG. 22, the fastener 34 can then be removed, allowing the base 20 to translate, right-to-left in FIG. 22, relative to the block 12 and/or the block 12 to translate left-to-right relative to the base 20.

As shown in FIG. 23, the block 12 and base 20 are relatively translated in a manner that the first and second bone parts 86, 88, fixed respectively to the block 12 and base 20, are moved so as to bring the facing bone surfaces 98, 100 closer together so as to reduce the width of the gap 96, or effectively eliminate the gap 96, to place the first and second bone parts 86, 88 in a desired end relationship, as shown in FIGS. 23-27. In each of these Figures, the facing surfaces 98, 100 are conformingly adjacent to, or against, each other.

In the form depicted, the first and second bone parts 86, 88 translate relative to each other in a path that is substantially straight and substantially parallel to the length of the bone 18 to effect shortening thereof.

In the depicted form, spaced undercuts 102 (FIG. 6) on the body 50 facilitate gripping by at least one clamp component 104 which can be manipulated to reposition the bone parts 86, 88, that can be done either manually or with the use of a clamping tool 106, as shown in FIG. 23.

With the desired relationship between the bone parts 86, 88 established, the through bore 72b on the body 50 aligns with the bore 76 in the base 20, whereby the fastener 34 can be threaded in place to maintain the aforementioned second relationship between the base 20 and block 12 and in turn the desired relationship of the bone parts 86, 88.

Once the FIG. 23 state for the system is arrived at, a guide block 32a, as shown clearly in FIGS. 15 and 16, with three guide passages 28a(1), 28a(2), 28a(3) therethrough, is placed in an operative position in a receptacle 108 (FIG. 6) on the block 12, as shown in FIGS. 24 and 25. As explained above with respect to FIG. 1, the guide block 32a, block 12, and base 20 might be alternatively configured so that the block 32a may engage/cooperate additionally with, or alternatively entirely with, the base 20, to be placed in its operative position. For purposes of simplicity in the claims herein, the guide block 32a will be considered to be a part of the block 12 and/or base 20 with which it is engaged.

The guide block 32a has a body 110 with oppositely facing, substantially parallel surfaces 112, 114 that respectively move guidingly against surfaces 116, 118 bounding the receptacle 108 to thereby cause the guide block 32a to be consistently guided in substantially the same orientation from a fully separated position, shown in dotted lines in FIG. 7, in the direction of the arrow 120 into an operatively engaged position. Consistent guiding results additionally from cooperation between oppositely facing width surfaces 122, 124 on the body 110 and facing surfaces 126, 128 bounding the receptacle 108.

The guide block 32a may be simply press fit and used, as contemplated for the depicted form. Enhanced holding, as by use of additional connecting/fastening structure, is also contemplated.

The leading surface 130 on the body 110 passes fully through the receptacle 108 to abut to the exposed bone surface 84. The surface 130 has an offset 132 to accommodate the bone contour at the site at which the system 10 is utilized. Alternatively, the guide block 32a may abut to the block 12 and/or base 20 when fully seated.

The guide passages 28a(1), 28a(2), 28a(3) successively have axes 134a(1), 134a(2), 134a(3) that define advancement lines for one or more fixation components 43 as well as drilling components 26 that may be utilized temporarily for fixation and to assist placement of the fixation components 43.

As shown in FIG. 24, the guide passages 28a(1), 28a(2), 28a(3) define alternative locations at which advancement lines can be established for the drilling components 26 and/or the fixation components 43.

Without limitation, a single drilling component 26 may be advanced through the guide channel 28a(2), or alternatively, separate drilling components 26 may be advanced through the guide passages 28a(1), 28a(3), as shown in FIG. 25. As depicted, each of the advancement lines extends through the second bone part 88, through the facing surfaces 98, 100, and into the first bone part 86. Once the drilling component(s) is inserted, its position can be checked and an appropriate length of a fixation component 43 determined. Making at least the block 12 and potentially additionally the base 20 from a non-metal material facilitates this length selection, as by permitting X-ray inspection. Without limitation, suitable materials are polyetheretherketone (PEEK) and polyeterimide (ULTEM).

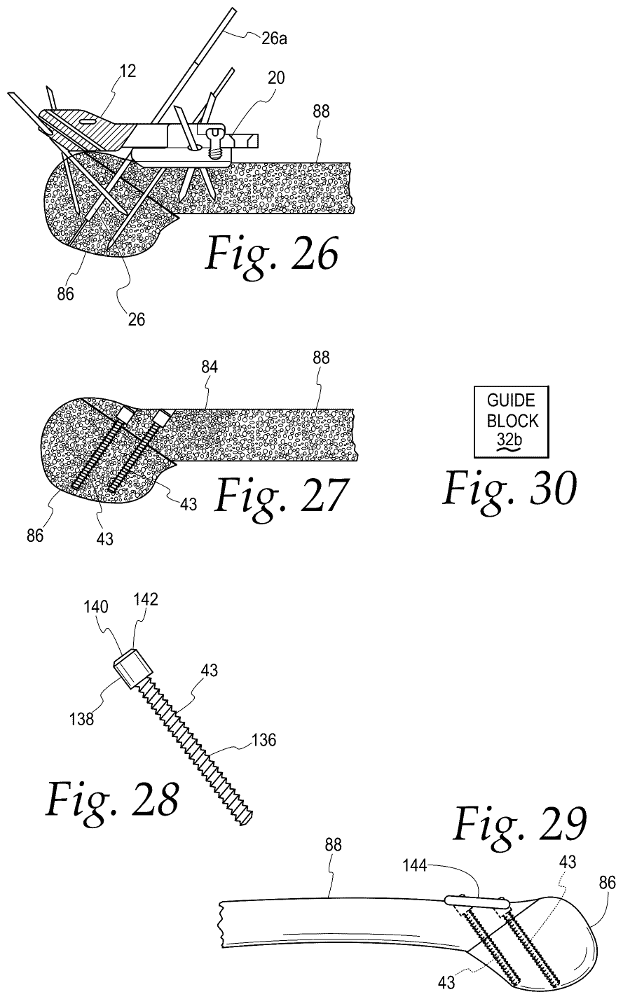

As shown in FIG. 26, the guide block 32a can then be separated, whereupon another drilling component 26a can be guided along the drilling components 26 embedded in the bone to produce one or more bores that will each accept a fixation component 43. With this arrangement, the bone parts 86, 88 remain stably held in the desired position with the receptacle 108 open to accommodate the drilling component 26a.

Once the bores have been formed through the drilling components 26a, the fixation components 43 can be directed into the formed bores as shown in FIG. 27.

In this embodiment, the fixation component 43 has a threaded shaft 136 and an enlarged head 138 through which the fixation component 43 can be turned. The fixation components may alternatively be, without limitation, headed screws, lag screws, headed compression screws, headless compression screws, etc.

Since a top surface 140 of the head will be angled with respect to the bone surface 84 through which it extends, an annular bevel 142 is formed on the head 138 to at least partially eliminate projection of a sharp edge at the bone surface 84 and have nominal conformity of the bevel angle to the surface 84, which permits a flush or near flush arrangement.

As shown in FIG. 29, an optional washer 144 may be provided through which the fixation components 43 extend. The washer may be made from an appropriate material, such as PEEK. Preferably, the configuration of the head 138 and washer 144 are such that the head 138 will engage the washer 144 through at least approximately 120° around the axis of the threaded shaft 136.

The guide block 32a is strategically designed taking into account the geometry of the particular bone and the relationship of the block 12 and base 20 therewith so that with the block 12 and base 20 in the operative relationship with each other and the bone 18, the advancement lines defined by the passages 28a(1), 28a(2), 28a(3) will be situated to establish a desired entry location and angle.

In the depicted form, the guide passages 28a(1), 28a(2), 28a(3) are located/oriented so that the axes 134a(1), 134a(2), 134a(3) are substantially parallel, equally spaced, and reside in a single plane. This orientation is not required, as the guide passages 28 may have different orientations and relationships.

Further, it is contemplated that only a single guide passage 28 may be provided.

Further, as shown in FIG. 30, a separate guide block 32b may be provided to be used interchangeably with the guide block 32a. The guide block 32b may have a different configuration than the guide block 32a, be it by reason of the orientation, number, relationship of one or more guide passages, etc. Thus, the surgeon has the option of selecting the block 32a or 32b, or from additional blocks, and performing a procedure, depending upon the site location and the approach to be taken during the procedure.

As noted previously, the removable and interchangeable guide blocks offer an option that is not required. The guide passages 28 may be formed directly on the block 12 and/or the base 20. In that case, the corresponding guide block is considered to be a part of the block 12 and/or base 20.

In one exemplary form, the block 12 may have a single piece that defines the guiding edges 14 as well as one or more guide passages 28.

It should be noted that the guiding edges 14 may be different than the specific form depicted. The guiding edges 14 may be angled so as to produce a wedge-shaped separable bone fragment. Additional guiding edges may be provided. As depicted, for the specific, exemplary application, the guiding edges 14 make an angle θ of approximately 25°-45° with the exposed bone surface 84 and preferably with the length of the bone 18.

As noted previously, the step of fixing the bone parts 86, 88, as identified schematically at the block 46 in FIG. 5, may be carried out in different manners, and using different structures/components.

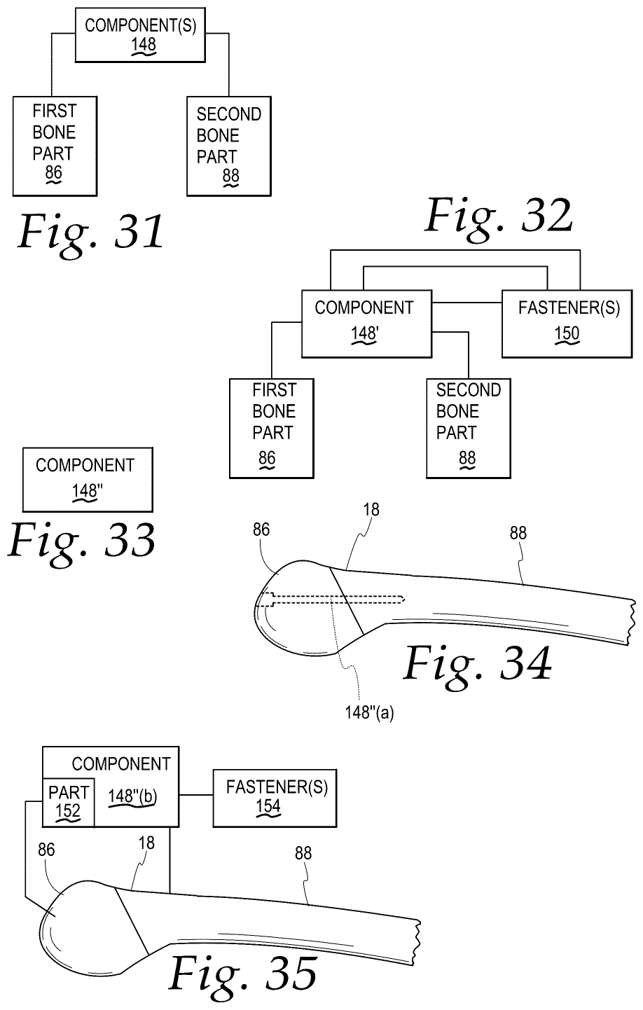

As shown schematically in FIG. 31, different forms of a component 148 may be fixed to each of the first and second bone parts 86, 88.

In one form, as shown schematically in FIG. 32, at least one fastener 150 is directed through the component 148′ and into each of the first and second bone parts 86, 88. In this form, the component 148′ may be in the form of a plate or another form that spans the bone parts 86, 88.

It is also possible that the component 148′ might be engaged with, and held against, one of the bone parts 86, 88 without a separate fastener.

The guide system 10 is strategically configured to allow the component 148′ to be placed and fixed through the fasteners 150 without interference from any other part of the guide system 10, including associated drilling components.

As shown schematically in FIG. 33, another form of the component 148″ is directed into each of the first and second bone parts 86, 88. This form encompasses the fixation component 43 and includes additional component configurations. As just one example, as shown in FIG. 34, the component 148″ (a) may extend through the first and second bone parts 86, 88 in a direction substantially parallel to the length of the bone 18. In one preferred form, the component 148′ (a) extends into the intramedullary space of at least one of the first and second bone parts 86, 88.

The bores 92a, 92b on the block 12 and bores 94a, 94b, 94c on the base 20 are strategically oriented so as not to intersect the space to be occupied by the component 148′″.

In yet another form of the component 148″, as shown schematically in FIG. 35 at 148″ (b), a part 152 of the component 148″ (b) is engaged with the first bone part 86 as by wrapping against a portion thereof, with or without penetration of the bone part 86, with another part of the component 148″ (b) fixed to the second bone part 88 through at least one fastener 154. The fastener(s) 154 may be directed through the another part of the component 148″ (b) and into the second bone part 88, or otherwise fixed thereto.

Different forms of the invention may be practiced using the block 12 and base 20 together, without any guide block 32 that may otherwise interfere with, for example, the use of the particular component 148.

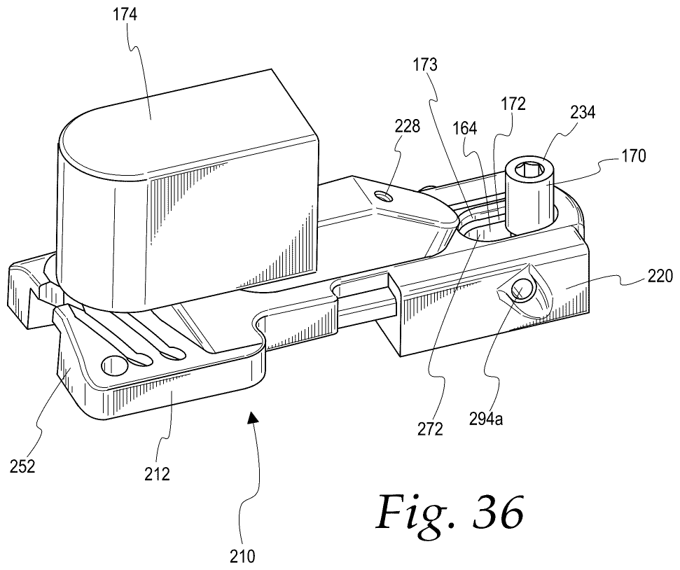

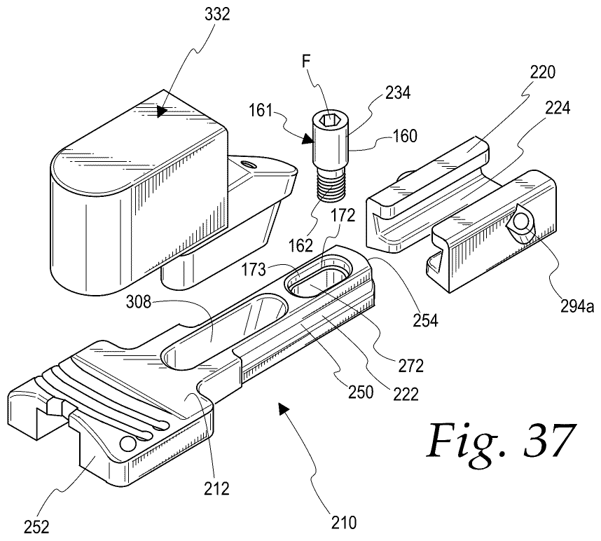

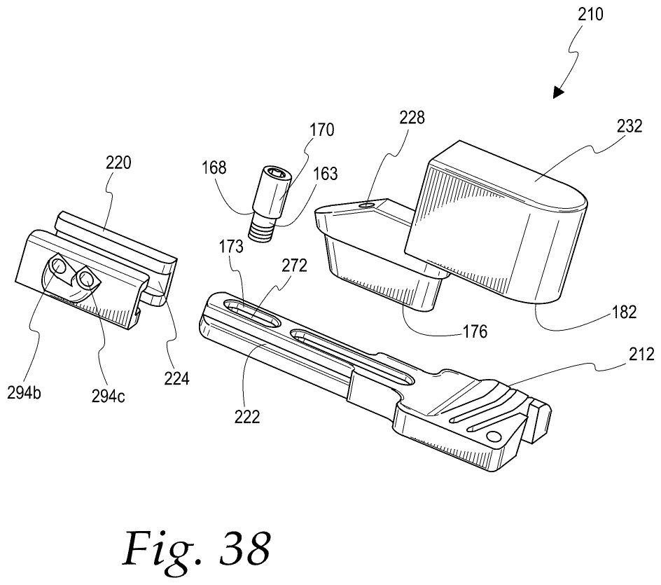

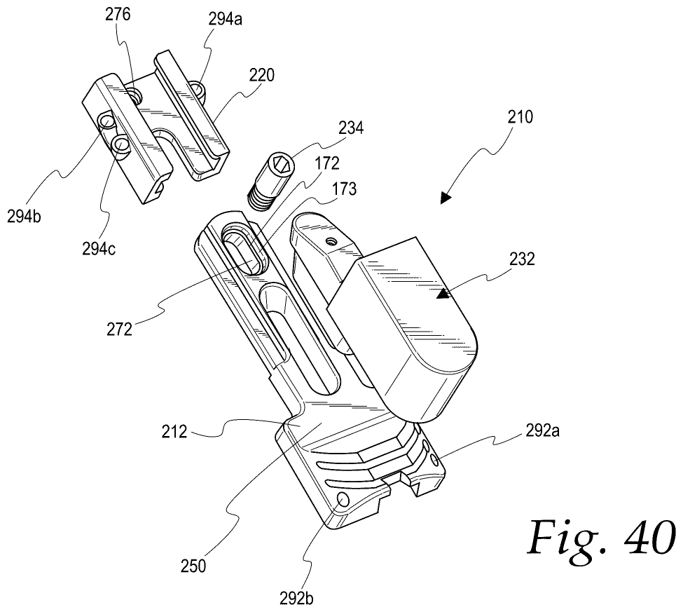

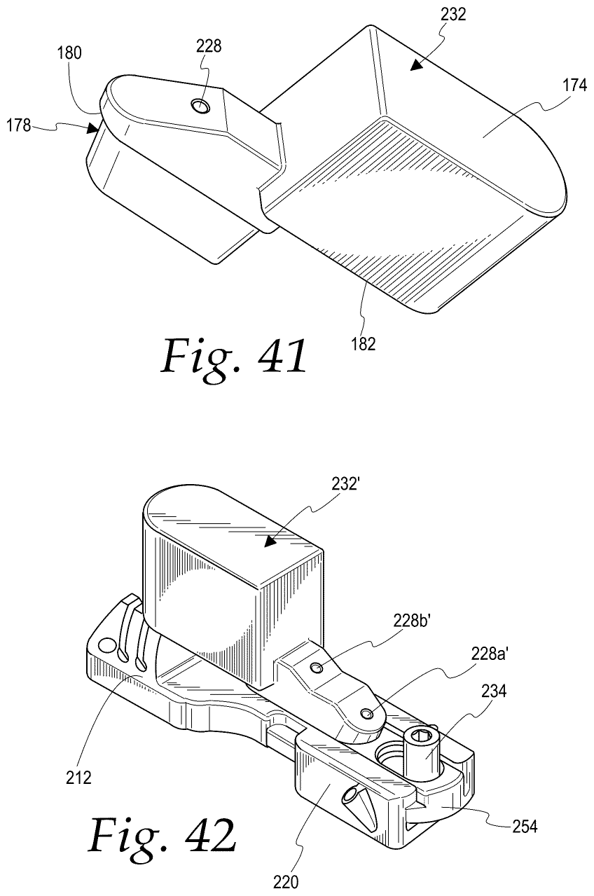

Another form of the inventive guide system is shown at 210 in FIGS. 36-41, with elements in FIGS. 36-41, corresponding to those in FIGS. 6-26, identified with the same reference numerals, with “200” added thereto. The guide system 210 is configured similarly to the guide system 10 and is used in substantially the same manner.

The block 212 has a body 250 with a length between spaced ends 252, 254 and has a width that is narrower at the end 254.

A connector 222 is provided at the narrower body end 254 with the guiding edges 214a, 214b adjacent the opposite body end 252.

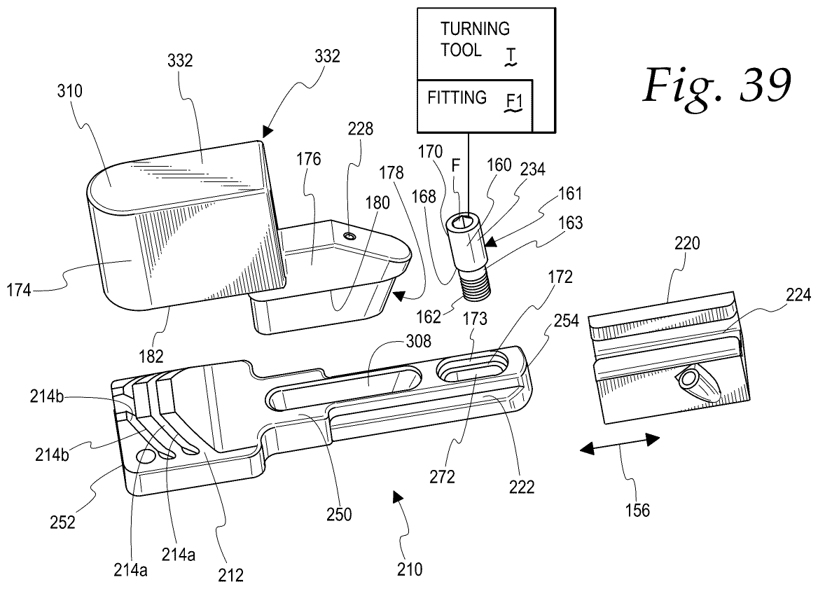

The connector 222 cooperates with a connector 224 on the base 220, making a keyed, rail-and-slot connection therewith through which the block 212 and base 220 are relatively guided in translation along the path indicated by the double-headed arrow 156 in FIG. 39.

Rather than providing the separate discrete receptacles 72a, 72b, a continuous elongate, oval, slot/receptacle 272 is formed through the body 250 to accept a fastener 234 directed therethrough and threadably engaged within a bore 276 in the base 220.

The fastener 234 has a body 160 with an unthreaded main part 161 having a stepped diameter and a threaded end 162 to cooperate with threads within the bore 276. A fitting F at the top of the fastener 234 facilitates turning with a tool T having a complementary fitting F1.

With the fastener 234 turned to be tightened within the bore 276 to a setup position, a smaller diameter portion 163 of the unthreaded main part 161 of the fastener 234 aligns with a guide surface 164 surrounding the slot 272. The portion 163 and the guide surface 164 may be configured to cooperate so that they, in conjunction with the connectors 222, 224, guide relative translational movement of the block 212 and base 220, whereupon the block 212 and base 220 can assume different relative positions corresponding to those in FIGS. 18 and 23. A portion of the threaded end 162 may also slide guidingly against the guide surface 164.

The ends of the slot 272 separately abut to the fastener 234 to establish the range of relative movement permitted between the block 212 and base 220 and may facilitate consistent establishment of two different, desired, operative relationships between the block 212 and base 220. A range of different operative relationships between these two operative relationships may also be maintained. In one form, the range of movement corresponds to the gap 96 resulting from separation of the bone fragment 90, whereby the gap 96 can be optimally closed between the facing surfaces 98,100.

The block 212 has through openings 292a, 292b to accept the aforementioned drilling components 26a, 26b, with the base 220 having openings 294a, 294b, 294c to accept at least the aforementioned drilling components 26c, 26d, whereby the base 220 and block 212 are fixed to respective bone parts 86, 88 so as to initially maintain the base 220 and block 212 in the aforementioned first operative relationship with each other and the bone 18.

The procedure is performed with the guide system 210 in substantially the same manner as it is performed with the guide system 10.

One difference between the guide systems 10, 210 is that with the guide system 210, the fastener 234 needs only to be loosened and re-tightened to selectively: a) allow relative movement between the block 212 and base 220; and b) releasably fix the base 220 and block 212 in different relationships; whereas with the guide system 10, the fastener 34 must be separated and re-inserted to maintain the desired different relationships between the base 20 and block 12.

To accomplish this, an annular shoulder 168, defined between the smaller diameter portion 163 and a larger diameter portion 170 on the fastener 234, aligns over and is abuttable to, a recessed upwardly facing, oval surface 172 adjacent the top of, and in the depicted form extending fully around, the slot 272. The shoulder 168 is fixedly held against the surface 172 with the fastener 234 tightened to thereby frictionally maintain the selected relationship between the base 220 and block 212. This configuration allows the base 220 and block 212 to be fixed over a range of selectable relative positions in the same manner and avoids having to separate, manipulate, and reinstall the fastener 234, as required for the fastener 34.

The larger diameter portion 170 of the fastener 234 has a substantial axial dimension that allows it to be comfortably grasped to facilitate preliminary placement, turning, separation, and handling of the fastener 234.

As depicted, the upwardly facing surface 172 is defined at a transition between the oval guide surface 164, surrounding the slot 272, and a larger oval surface 173 above the surface 172. The larger oval surface 173 may guide the larger diameter portion 170 of the fastener as the base 220 and block 220 relatively move.

Another difference between the guide systems 10, 210 relates to the corresponding guide blocks 32, 332. The guide block 332 has a body 310 with an enlarged grasping portion 174 that connects to an insertion portion 176 with a tapered bottom region 178 that is guided into a complementary receptacle 308 on the block 212. The tapered bottom region 178 is guided into the receptacle 308 with a circumferential shoulder 180 abutting to the top of the block 212 with the guide block 332 fully seated to thereby consistently establish an engaged/assembled/joined relationship between the guide block 332 and the block 212. The bottom 182 of the grasping portion 174 may simultaneously abut to the top of the block 212 to further stabilize the guide block 332 in its engaged/assembled/joined relationship.

The depicted form of the guide block 332 has a single guide passage 228 but could have multiple guide passages 228 as on the guide block 32 and/or a modified form of the guide block 332′ in FIG. 42, described below.

Multiple interchangeable guide blocks 332 may be kept on hand with different guide passage arrangements-different primarily in terms of number of guide passages 228, angular orientation of the guide passage 228, etc.

Since the inventive method is performed in substantially the same way with the guide system 210 as with the guide system 10, there is no need to herein describe the steps for performing the inventive method using the guide system 210.

The relatively large volume of the grasping portion 174 of the guide block 332 facilitates positive handling and stabilization of the guide block and associated block 212, which is particularly advantageous when advancing drilling components into and through the guide passage(s) 228.

As noted above, the guide block 332′ in FIG. 42 has multiple guide passes 228a′, 228b′, with it contemplated that more than the two guide passages 228a′, 228b′ might be incorporated. The guide block 332′ may otherwise have substantially the same configuration as the guide block 332 and may be usable in substantially the same manner with the same base 220 and block 212, or different forms thereof.

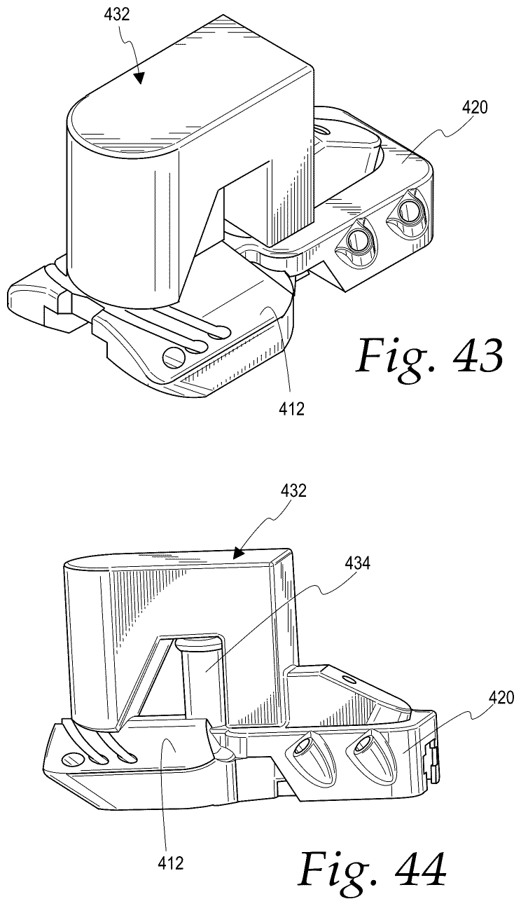

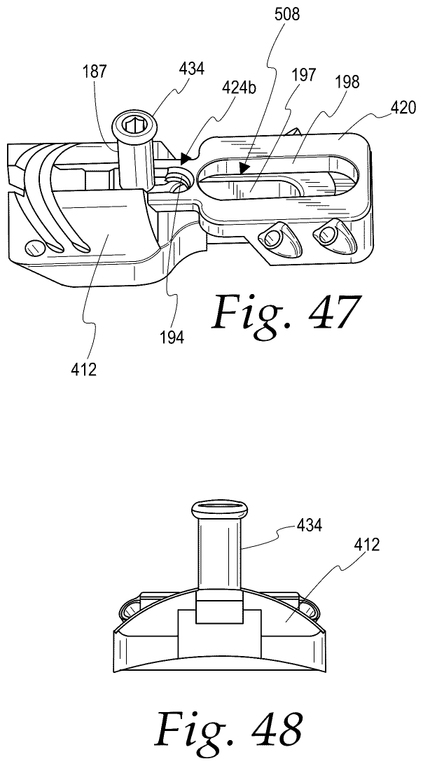

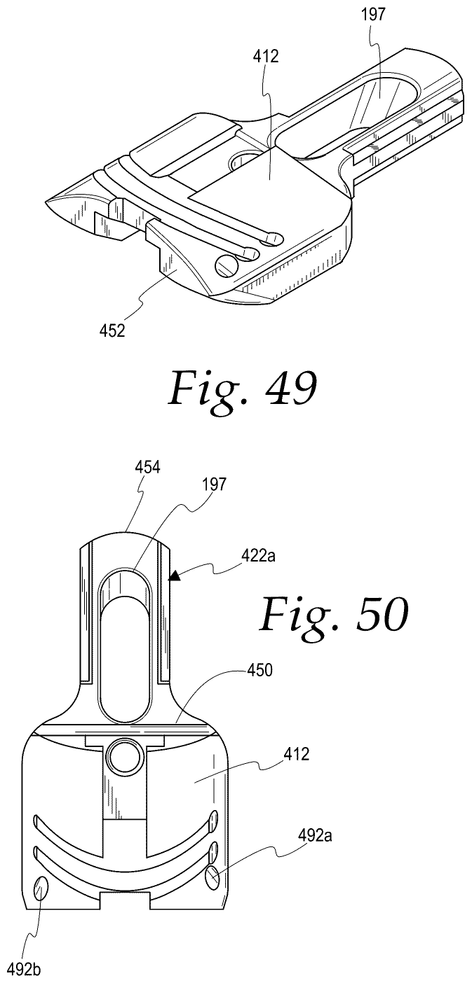

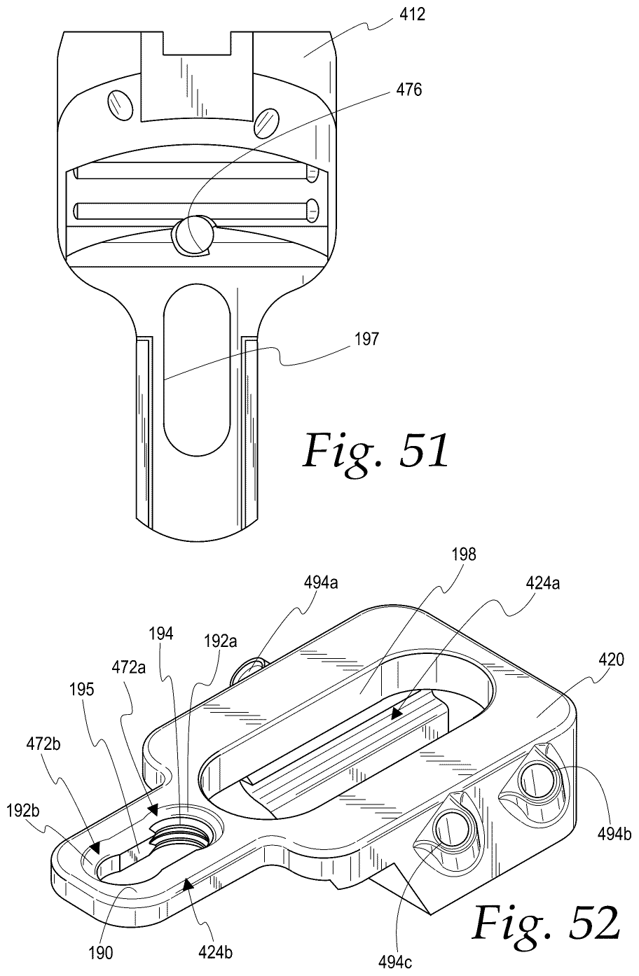

Another form of the inventive guide system is shown at 410 in FIGS. 43-52. The guide system 410 is configured similarly to the guide systems 10, 210 and is used in substantially the same manner. Elements in the guide system 410, corresponding to those in the guide system 10, will be used with the same reference numerals with “400” added.

The guide system 410 uses four basic components corresponding to those in the guide systems 10, 210-a block 412, a base 420, a fastener 434 cooperating between the base 420 and block 412, and a guide block 532.

As with the block 212 and base 220 on the guide system 210, at the start of a procedure, the block 412 and base 420 are fixed in a corresponding first operative relationship with each other and the bone 18.

The base 420 has openings 494a, 494b, 494c to guide drilling component 26 in a predetermined path through the base 420 and into the bone, where the bone part 88 will be defined. Fewer than the three exemplary openings 494 may be utilized.

The block 412 has openings 492a, 492b through which drilling components 26 are guided through the block 412 and into the bone, which becomes the bone part 86.

Accordingly, after the bone fragment is separated, the bone parts 86, 88 will guidingly move, together with the block 412 and base 420, respectively, to reduce the width of the gap 100, as seen in FIG. 21.

One difference between the guide system 410 and the guide system 210 is that the guide system 410 uses two rail-and-slot connections whereas the guide system 210 relies on a single rail-and-slot connection.

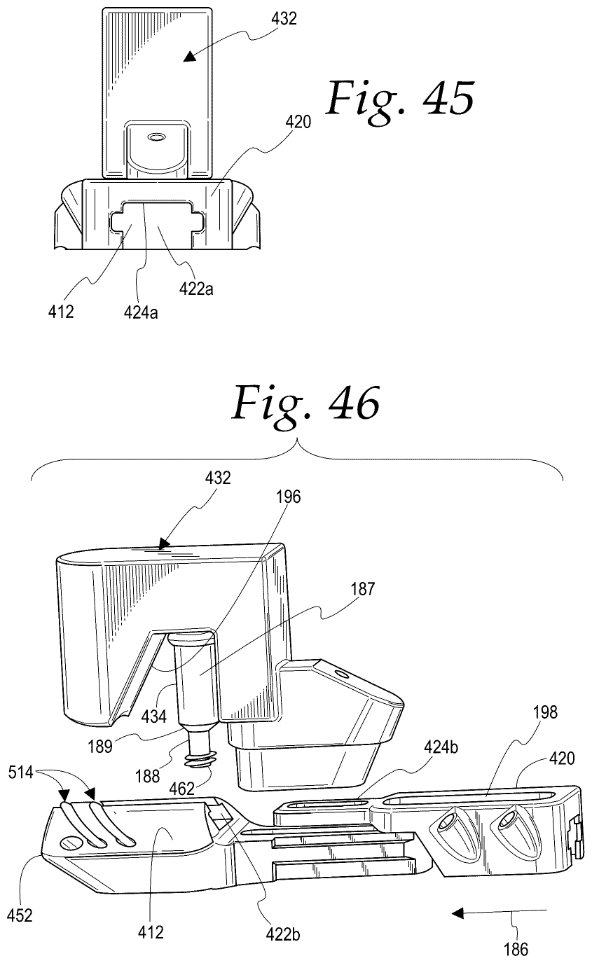

More specifically, the base 420 has a slot connector 424a that cooperates with a rail connector 422a on the block 412. As seen clearly in FIG. 45, the rail connector 422a has a T-shaped cross-sectional configuration which slides within a matching volume of the slot connector 424a.

The cooperating connectors 422a, 424a interact adjacent one end 454 of the body 450 of the block 412.

The base 420 additionally has a rail connector 424b that cooperates with a slot connector 422b on the block 412.

With the base 420 and block 412 in the FIG. 46 relationship, and the connectors 422a, 424a; 422b, 424b aligned, the base 420 can be advanced in the direction of the arrow 186 relative to the block 412, and/or the block 412 can be advanced oppositely relative to the base 420, to thereby simultaneously engage both connector pairs 422a, 424a; 422b,424b. The two connector pair arrangement stabilizes the connection between the base 420 and block 412 and contributes to smoother and more precise guided movement between the base 420 and block 412.

Another difference between the guide system 410 and the guide system 210 relates to the location of the fastener 434 that cooperates between the base 420 and block 412. As seen in FIG. 42, the fastener 234 on the guide system 210 is adjacent to the end 254 of the body 250, whereas the fastener 434 on the guide system 410 is closer to the end 452 of the corresponding body 450. As a result, with the guide system 210, a longer incision would have to be made through the tissue overlying the bone 18 to accommodate the projecting fastener 234.

On the other hand, the fastener 434 is in closer proximity to the guiding edges 414 whereat exposure to the bone 18 is already required to form and extract the bone fragment.

Further, the fastener 434 cooperates with the base 420 and block 412 in a different manner than the fastener 234 cooperates with the corresponding base 220 and block 212.

The fastener 434 has a stepped diameter with a graspable larger diameter portion 187 and a smaller diameter portion 188, with the smaller diameter portion terminating at a threaded end 462. A beveled transition region 189 resides between the larger diameter portion 187 and smaller diameter portion 188 of the fastener 434.

The base 420 has a cantilevered part 190 on which the connector 424b is formed. Spaced receptacles 472a, 472b are provided through the cantilevered part 190 and, while on the base 420, correspond in function to the receptacles 72a, 72b on the block 12 on the guide system 10. The cantilevered part 190 is cut away so that the smaller diameter portion 188 of the fastener 434, when directed into either of the receptacles 472a, 472b, can be shifted into the other of the receptacles 472a, 472b. Beveled surfaces 192a, 192b, while interrupted, have adequate circumferential extent to each stably seat the beveled region 189 of the fastener 434.

The fastener 434 is initially engaged with the base 420 by engaging threads on the threaded end 462 of the fastener 434 with threads 194 around the receptacle 472a. By advancing the threaded end 462 fully through the receptacle 472a to disengage the threads, the fastener 434 is prevented from separating from the cantilever part 190 other than by reengaging the threads 194 and reversely rotating the fastener 434.

The threaded end 462 of the fastener 434, upon being directed fully through the receptacle 472a, is threadably engagable with a threaded bore 476 on the block 412. The threaded bore 476 corresponds in function to the threaded bore 76 on the base 20 on the guide system 210.

With the connector pairs 422, 424 engaged, the receptacles 472a, 472b can be selectively axially aligned with the threaded bore 476.

The base 420 and block 412 are in the aforementioned first operative relationship with the receptacle 472b axially aligned with the bore 476 and in the aforementioned second operative relationship with the receptacle 472a axially aligned with the bore 476.

With the receptacle 472b aligned with the bore 476, the smaller diameter portion 188 of the fastener 434 extending through the receptacle 472b, and the threaded fastener end 462 threadably engaged within the bore 476, tightening of the fastener 434 draws the beveled region 189 against the beveled surface 192b to positively fix the base 420 and block 412 against relative translational movement.

By thereafter loosening the fastener 434 while maintaining the fastener end 462 threadably engaged within the bore 476, the base 420 and block 412 can be relatively moved to align the receptacle 472a with the threaded bore 476. As this occurs, the smaller diameter portion 188 of the fastener 434 is allowed to translate through a connecting space 195, communicating between the receptacles 472a, 472b, to reside in the receptacle 472a. By then retightening the fastener 434, the beveled region 189 can be drawn against the beveled surface 192a to positively maintain the changed relationship between the base 420 and block 412.

Another difference between the guide system 410 and the guide system 210 is that the guide block 532, which is made to function substantially the same as the guide block 232, has a cutout 196 to accommodate the graspable upwardly projecting larger diameter portion 187 of the fastener 434.

The cutout 196 might be eliminated by making the top of the fastener 434 flush with the top of the block 412. This, however, precludes comfortable hand grasping of the fastener 434 by eliminating the upwardly projecting length thereof.

The guide block 532 may have a slotted opening to accommodate the movement of the graspable upwardly projecting larger diameter portion 187 of the fastener 434.

Another difference is that the receptacle 508, corresponding to the receptacle 308 on the guide system 210, which receives the tapered bottom region 178 in the guide system 210, is defined cooperatively by oval surfaces 197, 198, respectively on the block 412 and base 420, that register with each other with the base 420 and block 412 in the second operative relationship.

As described with respect to earlier embodiments, one or both of the block 412 and base 420 may be made from a non-metal material, such as PEEK or ULTEM, to among other things, facilitate using X-ray to control depth of bone penetration by the drilling components 26.

It should be emphasized that throughout, the many different forms of components and their interactions described are exemplary in nature only, with different forms and relationships contemplated. As just one example, the male/female relationship between parts on the rail-and-slot connections may be reversed.

The foregoing disclosure of specific embodiments is intended to be illustrative of the broad concepts comprehended by the invention.

1. A method of shortening a bone, the method comprising the steps of:
obtaining a guide system comprising a block and a base;
releasably fixing the block and base in a first operative relationship with each other and the bone;
with the block and the base in the first operative relationship with each other and the bone, using the block to guide a cutting component relative to the bone so as to cut the bone to thereby form a separable bone fragment between first and second relatively movable bone parts;
separating the bone fragment from the bone so that a gap is defined between facing surfaces on the first and second relatively movable bone parts,
whereby with the block and base fixed in the first operative relationship with each other and the base: a) the block is fixed to a part of the bone, that becomes the first bone part, through at least a first fixation component; and b) the base is fixed to a part of the bone, that becomes the second bone part, through at least a second fixation component;
after separating the bone fragment, relatively moving the base and block to thereby cause the first and second bone parts to relatively move so that the facing surfaces on the first and second bone parts are moved closer to each other to thereby place the first and second bone parts in a desired relationship;
fixing the first and second bone parts in the desired relationship; and
separating the block and base from the bone.

2. The method of shortening a bone according to claim 1 wherein the step of fixing the first and second bone parts comprises fixing a component to each of the first and second bone parts.

3. The method of shortening a bone according to claim 2 wherein the step of fixing the first and second bone parts comprises directing at least one fastener through the component and into each of the first and second bone parts.

4. The method of shortening a bone according to claim 1 wherein the step of fixing the first and second bone parts comprises engaging a part of a component with the first bone part and directing at least one fastener through the component and into the second bone part.

5. The method of shortening a bone according to claim 1 wherein the step of fixing the first and second bone parts comprises directing a fixation element into the first and second bone parts.

6. The method of shortening a bone according to claim 5 wherein the bone has a length and the step of directing a fixation element comprises directing a first fixation element lengthwise of the bone into each of the first and second bone parts.

7. The method of shortening a bone according to claim 6 wherein the step of directing a first fixation element lengthwise of the bone comprises directing the fixation element through an intramedullary space on at least one of the first and second bone parts.

8. The method of shortening a bone according to claim 5 wherein the step of directing a fixation element into the first and second bone parts comprises directing at least a first fixation element through the facing bone surfaces.

9. The method of shortening a bone according to claim 5 wherein the bone is one of an ulna bone and a second metatarsal bone.

10. The method of shortening a bone according to claim 5 wherein the guide system further comprises a first guide block defining at least a first guide passage and the method further comprises the step of directing a drilling component through the first guide passage to define an advancement line for the first fixation element.

11. The method of shortening a bone according to claim 10 further comprising the step of operatively engaging the first guide block with at least one of the block and base.

12. The method of shortening a bone according to claim 5 wherein the guide system further comprises first and second different guide blocks each defining a guide passage and further comprising the steps of operatively engaging a selected one of the first and second different guide blocks with at least one of the block and base and thereafter directing a drilling component through the guide passage on the selected one of the first and second different guide blocks to define an advancement line for the first fixation element, the first and second different guide blocks being interchangeably usable, one in place of the other.

13. The method of shortening a bone according to claim 5 wherein the guide system further comprises a first guide block defining first and second guide passages and the method further comprises the step of directing a first drilling component through one of the first and second guide passages to define an advancement line for the first fixation element.

14. The method of shortening a bone according to claim 5 wherein the block has first and second spaced guiding edges which guide at least one of: a) a part of a cutting tool having the cutting component; and b) the cutting component.

15. The method of shortening a bone according to claim 14 wherein there is a single piece that defines the first and second guiding edges and a guide passage that defines an advancement line for the first fixation element.

16. The method of shortening a bone according to claim 5 wherein the block has a connector that cooperates with a connector on the base, and the step of relatively moving the base and block comprises guidingly moving the base and block through the cooperating connectors on the base and block.

17. The method of shortening a bone according to claim 16 wherein the connector on one of the block and base is in the form of a rail that moves guidingly within a slot making up the connector on the other of the block and base such that the block and base are relatively movable along a substantially straight line and are keyed against turning angularly relative to each other around the substantially straight line.

18. The method of shortening a bone according to claim 16 wherein the step of fixing the first operative relationship between the block and base comprises using a fastener cooperating between the block and base.

19. The method of shortening a bone according to claim 18 further comprising the step of fixing a second relationship between the block and base through a fastener cooperating between the block and base with the first and second bone parts in the desired relationship.

20. The method of shortening a bone according to claim 19 wherein the fastener that cooperates between the block and base with the block and base in the first operative relationship is a same fastener that cooperates between the block and base with the block and base in the second relationship, and is a threaded fastener that is directed into: a) a first receptacle on the block with the block and base in the first operative relationship with each other and the bone; and b) a second receptacle on the block with the block and base in the second relationship.

21. The method of shortening a bone according to claim 19 wherein the fastener that cooperates between the block and base with the block and base in the first relationship is a same fastener that cooperates between the block and base with the block and base in the second relationship and is a threaded fastener that is directed into a first receptacle on the block with: a) the block and base in the first operative relationship; and b) with the block and base in the second relationship.

22. The method of shortening a bone according to claim 21 wherein the threaded fastener extends through: a) a first receptacle on the block with the block and base in the first operative relationship; and b) a second receptacle on the block with the block and base in the second relationship.

23. The method of shortening a bone according to claim 19 wherein the step of releasably fixing the block and base in the first operative relationship comprises tightening the fastener, and further comprising the step of loosening the tightened fastener with the block and base in the first operative relationship and without separating the fastener from the base, after loosening the tightened fastener, relatively moving the block and base from the first operative relationship into the second relationship, and re-tightening the fastener to fix the second relationship between the block and base.

24. The method of shortening a bone according to claim 16 wherein the block has another connector that cooperates with another connector on the base and each of the: a) cooperating connectors; and b) cooperating another connectors is in the form of a rail that moves guidingly within a slot.

25. The method of shortening a bone according to claim 24 wherein the bone has a length and the cooperating connectors are spaced from the cooperating another connectors along the length of the bone.

26. The method of shortening a bone according to claim 8 wherein the step of relatively moving the base and block comprises using a clamping device to facilitate relative movement of the first and second bone parts so as to thereby urge the facing bone surfaces towards each other.

27. The method of shortening a bone according to claim 5 wherein the step of releasably fixing the block and base in the first operative relationship comprises directing first and second drilling components through the block into the bone.

28. The method of shortening a bone according to claim 5 wherein the step of releasably fixing the block and base comprises directing first and second drilling components through the base into the bone.

29. The method of shortening a bone according to claim 5 wherein the bone has a length and the step of relatively moving the base and block comprises causing the first and second bones to relatively move along a path substantially parallel to the length of the bone.

30. The method of shortening a bone according to claim 19 wherein the fastener guides movement between the block and base as the block and base are changed between the first operative relationship and the second relationship.

31. The method of shortening a bone according to claim 14 wherein the bone has a surface against which the block is placed with the block and base in the first operative relationship with each other and the bone, the first and second guiding edges are substantially parallel, and the guiding edges make an angle of 25°-45° with respect to a length of the bone.

32. The method of shortening a bone according to claim 5 wherein the bone fragment has a substantially uniform thickness.

33. The method of shortening a bone according to claim 5 wherein with the block and base in the first operative relationship with each other and the bone, the block bears directly against the first bone part and the base bears directly against the second bone part.

34. The method of shortening a bone according to claim 33 wherein the base has first and second spaced, elongate, depending portions that each bears against the bone with the block and base in the first operative relationship with each other and the bone.

35. The method of shortening a bone according to claim 21 wherein the block has first and second spaced guiding edges which guide at least one of: a) a part of a cutting tool having the cutting component; and b) the cutting component, the block has spaced ends, and each of: i) the first and second edges on the block; and ii) the first receptacle on the block are closer to a same one of the spaced ends on the block.

36. A guide system for performing an osteotomy on a bone, the guide system comprising:
a block defining first and second edges each for guiding a cutting component in a controlled path,
the block configured to be placed in an operative position against the surface of the bone and to be releasably fixed in the operative position; and
a base configured to be placed in an operative position against the surface of the bone and to be releasably fixed in its operative position,
the block having a connector that cooperates with a connector on the base to guide relative movement between the base and block substantially along a first line,
the block and base configured so that in respective operative positions a cutting component can be guided through the first and second edges to produce a separable bone fragment,
whereupon separation of the bone fragment produces a gap of a first width between facing surfaces on relatively movable bone parts,
the block and base configured so that with the bone fragment separated the block and base can be guidingly relatively moved to reduce the width of the gap from the first width, move the facing surfaces towards each other, and place the relatively movable bone parts in a desired relationship.

37. The guide system for performing an osteotomy according to claim 36 in combination with at least one drilling component that can be directed through the block and into the bone to maintain the block in its operative position.

38. The guide system for performing an osteotomy according to claim 36 in combination with at least one drilling component that can be directed through the base and into the bone to maintain the base in its operative position.

39. The guide system for performing an osteotomy according to claim 36 wherein the guide system further comprises a fastener cooperating between the base and block to selectively releasably maintain the base and block in first and second different relative positions.

40. The guide system for performing an osteotomy according to claim 39 wherein the fastener is a threaded fastener that can be directed into at least one receptacle on the block.

41. The guide system for performing an osteotomy according to claim 40 wherein the block has a single receptacle for the threaded fastener and the base has first and second receptacles through which the threaded fastener is selectively directed and into the single receptacle on the block, the guide system configured so that the threaded fastener can be: a) loosened without being separated from the base to allow the block and base to be relatively moved between the first and second different relative positions wherein the threaded fastener is respectively directed through the first and second receptacles on the base; and b) re-tightened to releasably maintain the block and base in the first and second different relative positions.

42. The guide system for performing an osteotomy according to claim 36 wherein the cooperating connectors are on the block and base and make up a rail and slot connection whereby the block and base are guided relative to each other.

43. The guide system for performing an osteotomy according to claim 42 wherein the block has another connector that cooperates with another connector on the base and each of the: a) cooperating connectors; and b) cooperating another connectors is in the form of a rail that moves guiding within a slot.

44. The guide system for performing an osteotomy according to claim 43 wherein the bone has a length and the cooperating connectors are spaced from the cooperating another connectors along the length of the bone.

45. The guide system for performing an osteotomy according to claim 36 wherein there is a guide passage on at least one of the block and base that defines an advancement line for a fixation element that can be directed through the bone parts and the facing bone surfaces to maintain the bone parts in the desired relationship.

46. The guide system for performing an osteotomy according to claim 45 wherein the block has a body that defines the first and second edges and the guide passage.

47. The guide system for performing an osteotomy according to claim 45 wherein the guide passage is defined on a first guide block that is configured to be releasably engaged with at least one of the block and the base.

48. The guide system for performing an osteotomy according to claim 47 in combination with a second guide block with a guide passage that is different than the first guide block and that is configured to be releasably engaged with at least one of the block and the base, the first and second guide blocks interchangeably usable, one in place of the other.

49. The guide system for performing an osteotomy according to claim 48 wherein with the first and second guide blocks engaged with the at least one of the block and the base, the guide passages on the first and second guide blocks are different by defining advancement lines having at least one of: a) different orientations; and b) different entry locations on the bone.

50. The guide system for performing an osteotomy according to claim 36 in combination with a component usable to maintain the bone parts in the desired relationship.

51. The guide system for performing an osteotomy according to claim 50 wherein the component is configured to be directed into an intramedullary space of one of the first and second bone parts.

52. The guide system for performing an osteotomy according to claim 39 wherein the fastener is configured to guide relative movement of the base and block between the first and second different relative positions.

53. The guide system for performing an osteotomy according to claim 36, in combination with at least one drilling component configured to be directed through the base and into a bone to releasably fix the base in its operative position.

54. The guide system for performing an osteotomy according to claim 36 in combination with at least one drilling component configured to be directed through the block and into a bone to releasably fix the block in its operative position.
